(19) United States
(12) Patent Application Publication
(10) Pub. No.: US 2006/0190034 A1
(43) Pub. Date: Aug. 24, 2006

(54) Surgical instrument

(76) Inventors: Kouji Nishizawa, Hitachinaka (JP); Takeshi Hoshino, Kodaira (JP); Yukinobu Maruyama, Kokubunji (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/362,140

(22) Filed: Feb. 27, 2006

(30) Foreign Application Priority Data
Oct. 25, 2004 (JP) 2004-309613

(51) Int. Cl. A61B 17/28

(52) U.S. Cl. 606/205

(57) ABSTRACT

A surgical instrument comprising a tip end joint part having an openable and closeable gripper, an operating part including a hand grip and a plurality of operating dials, and an arm part that accommodates a wire for cooperation of actions of the operating part and the tip end joint part. A first operating dial is arranged above the hand grip and on an upper inclined surface of the operating part, second and third operating dials are arranged above the hand grip and on a front surface of the operating part, the first operating dial is operated by a thumb and the second operating dial is operated by a forefinger whereby the tip end joint part is operated vertically and operated laterally to perform a swinging action, and the third operating dial is operated by a forefinger whereby the tip end gripper is operated to open and close.

### BACKGROUND OF THE INVENTION

The present invention relates to a surgical instrument to be used in a clinical site, and more particular, to a surgical instrument, of which a position and a posture can be operated by a wire drive type joint.

As a related art for a medical manipulator, which includes movable parts of two degrees of freedom at a tip end of a forceps and a forceps operating part at a rear end of the forceps to minutely operate a forceps, JP-A-2001-276091 discloses an arrangement, which comprises at a tip end of a forceps a first rotary joint and a second rotary joint, and in which a motor at an operating part drives to control the respective joints through gears, etc. to perform an operation of determining a posture of a tip end of the forceps, and a lever provided on an operating rod is operated to control an amount of opening and closing of the forceps.

JP-A-2004-154164 discloses a related art of a multiple degree-of-freedom type treatment tool including a treatment tool body comprising a treatment part connected thereto through an joint, a joy stick, which bends the treatment part in a vertical direction and in a lateral direction, a dial, which rotationally operates the treatment part, and a lever, which operates the treatment part to open and close the same, wherein the treatment part is simply set in a desired position and a desired posture.

There is disclosed a related art for a medical manipulator, in which miniaturization and controllability of the manipulator are improved by maintaining drive wires in path length and phase irrespective of an angular change in joints (see, for example, JP-A-2004-122286).

With the related art disclosed in JP-A-2001-276091, however, a complicated control of rotary operations of first and second rotating shafts provided on the joints must be exercised by operation of the joy stick and driving of the motor since the joy stick provided on the operating rod is operated lateral and vertically in a complicated manner to determine a position and a posture of the forceps and determination of the position and the posture is made by driving a motor provided on the operating rod.

With the disclosure of JP-A-2004-154164, any motor is not used but a joy stick for manual operation is used to operate drive wires directly, so that skill is necessary in operation of the joy stick when appropriately setting the treatment part in position and posture. With the construction of joints proposed in JP-A-2004-122286, control of a motor is involved in a method of driving swinging movements and opening and closing movements of the blades in the embodiment disclosed therein.

It is an object of the invention to provide a surgical instrument, a position and a posture of a multiple degree-of-freedom gripper of which can be easily manipulated by movements without strain of a wrist and fingers of an operator at an operating part.

### BRIEF SUMMARY OF THE INVENTION

According to an aspect of the invention, there is provided a surgical instrument comprising a tip end joint part having an openable and closeable gripper, an operating part including a hand grip to be grasped by a palm and a plurality of operating dials, and an arm part that accommodates wires for cooperation of actions of the operating part and the tip end joint part, and wherein a first operating dial is arranged above the hand grip and on an upper inclined surface of the operating part and second and third operating dials are arranged above the hand grip and on a front surface of the operating part, and wherein the first operating dial is operated by a thumb and the second operating dial is operated by a forefinger whereby the tip end joint part is operated vertically and laterally to perform a swinging action, and the third operating dial is operated by a forefinger whereby the tip end gripper is operated to open or close.

According to another aspect of the invention, there is provided a surgical instrument comprising a tip end joint part having an openable and closeable gripper, an operating part including a hand grip to be grasped by a palm and a plurality of operating dials, and an arm part that accommodates wires for cooperation of actions of the operating part and the tip end joint part, and wherein the operating part is shaped to be schematically modified-elliptical in cross section, first and second operating dials are arranged on an inclined surface formed on an upper portion of the hand grip on this side of the operating part, and a third operating dial is arranged on the upper portion of the hand grip on an opposite side to this side, and wherein the first and second operating dials are operated by a thumb to move the tip end joint part vertically and laterally to have the tip end joint part swing, and the third operating dial is operated by a forefinger whereby the tip end gripper is operated to open and close.

According to the invention, a position and a posture of a gripper, which functions as a forceps, can be easily and stably operated by an operator movements without strain at an operating part without the use of electronic control such as an actuator, etc.

Since a position and a posture of a gripper can be easily operated mainly by a thumb and a forefinger at an operating part, the surgical instrument is suited to an operation during a long period of time. Also, it is possible to provide a surgical instrument, which is simple in construction and operation.

Other objects, features, and advantages of the invention will be made apparent from the following descriptions with respect to an embodiment of the invention.

### BRIEF DESCRIPTION OF THE SEVERAL VIEWS OF THE DRAWING

FIG. 1 is a perspective view of a tip end portion of a surgical instrument according to the invention;

FIG. 2 is an exploded perspective view of the tip end portion shown in FIG. 1 with wires, which drive swing of blades and joints, being omitted for easy comprehension;

FIGS. 3A and 3B are views to describe a wiring state of wires at the tip end portion and showing joints in a straight state and in a bent state, respectively;

FIG. 4 is a detailed perspective view of the tip end portion;

FIGS. 5A and 5B are perspective views showing a whole construction of a first modification of a surgical instrument according to the invention;

FIGS. 6A and 6B are perspective views showing a state, in which the first modification is grasped by a right hand;

FIG. 7 is a view showing an arrangement of an operating dial at a hand grip of the first modification;

FIG. 8 is a perspective view showing mounting and dismounting of a stability holder on the hand grip of the first modification;

FIGS. 9A, 9B, and 9C are views illustrating a state, in which drive wires are stretched between an joint mechanism at a tip end and an operating mechanism on the hand side in the first modification;

FIGS. 10A and 10B are perspective views showing a detailed construction of a lateral swing dial in the first modification;

FIGS. 11A and 11B are perspective views showing a whole construction of a second modification of a surgical instrument according to the invention;

FIGS. 12A and 12B are perspective views showing a whole construction of a third modification of a surgical instrument according to the invention;

FIGS. 13A and 13B are perspective views showing a state, in which an operating part of the third modification is grasped by a right hand;

FIGS. 14A, 14B, and 14C are a front view, a side view, and a plan view of the operating part of the third modification, respectively;

FIG. 15 is a view illustrating a state, in which drive wires are stretched between an joint mechanism at a tip end and an operating mechanism on the hand side in the second modification;

FIG. 16 is a plan view illustrating a wiring state of wires around an operating dial shown in FIG. 15;

FIG. 17 is a view illustrating a state, in which drive wires are stretched between an joint mechanism at a tip end and an operating mechanism on the hand side in the third modification; and

FIG. 18 is a plan view illustrating a wiring state of wires around the operating dial shown in FIG. 17.

### DETAILED DESCRIPTION OF THE INVENTION

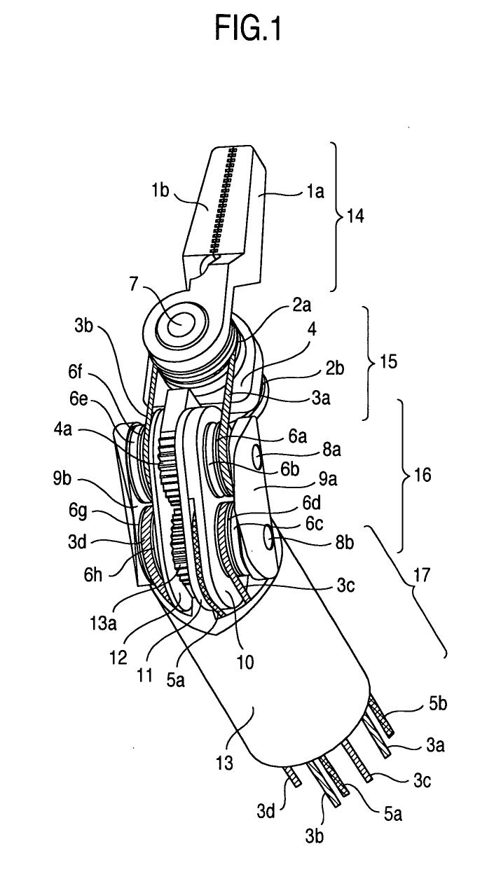

A surgical instrument according to the invention will be described with reference to FIGS. 1 to 4.

A surgical instrument for medical care will be exemplarily described for the purpose of a specific illustration (the invention is not specifically limited to a surgical instrument for medical care but provides a construction for general operation tools, a gripper of which is manually operated). A tip end portion (also, referred below to as a tip end joint or an instrument joint) of a surgical instrument (also, referred below to as instrument) comprises a gripper (forceps part) 14 that grips a suture thread, a needle, or the like, a tip end part 15 positioned near a lower portion of the gripper 14 shown in FIG. 1, an intermediate part 16 that forms a second joint together with the tip end part 15, a root part 17 that forms a first joint together with the intermediate part 16, and drive wires 3a to 3d, 5a, 5b for operation of the gripper 14, the tip end part 15, and the intermediate part 16. The tip end part functions as an joint of the instrument in the form of a forceps. The surgical instrument comprises, in addition to the tip end part, a hand side operation part (details of which are described later) not shown in FIG. 1 and disposed on a hand side of the root part 17 to operate pulling length of the drive wires 3a to 3d, 5a, 5b.

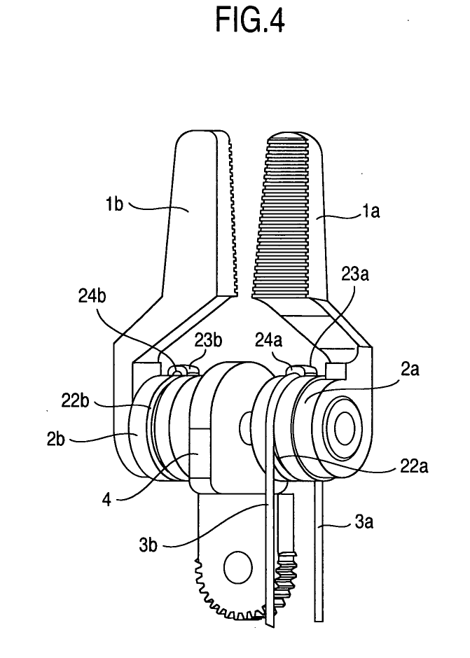

The gripper 14 comprises a pair of blades 1a, 1b, and blade pulleys 2a, 2b are arranged at roots of the respective blades 1a, 1b. The blade pulleys 2a, 2b are formed with grooves 22a, 22b, around which the drive wires 3a to 3d for operation of the blades are wound (see FIG. 4), and provided with holding portions 23a, 23b, by which the drive wires 3a to 3d wound around the grooves 22a, 22b are held on the blade pulleys 2a, 2b.

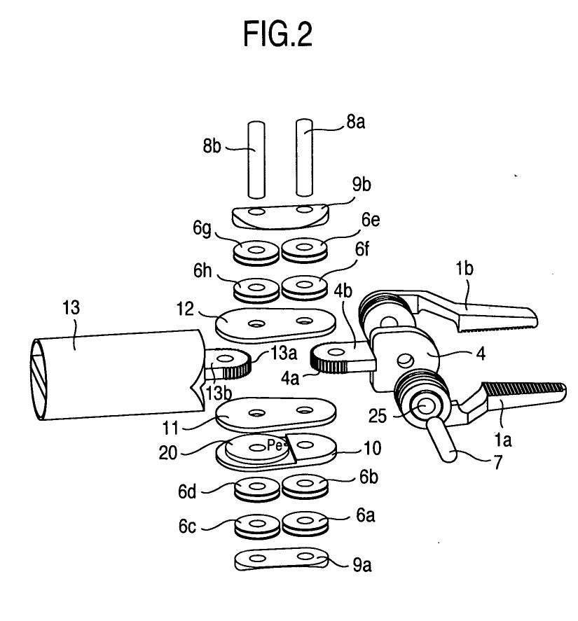

The tip end part 15 comprises a tip end base portion 4 in the form of a flat plate interposed between the pair of blades 1a, 1b, and a rolling member 4b being a flat plate substantially perpendicular to the tip end base portion 4 and having a semi-circular gear portion 4a (see FIG. 2). A hole is formed centrally of the tip end base portion 4, and a shaft 7 extends through the hole and holes formed centrally of the blade pulleys 2a, 2b. A hole is also formed centrally of the gear portion 4a, and a shaft 8a extending through the hole extends through a hole formed on the intermediate part 16.

The root part 17 comprises a cylindrical-shaped cylinder portion 13 and a rolling member 13b positioned at a tip end of the cylinder portion 13 and formed with a semi-circular gear portion 13a. A hole is formed centrally of the rolling member 13b (see FIG. 2). The semi-circular gear portions 4a, 13a serves as means for rolling contact, and in addition to measures for use of gear portions, there are measures such as working for an increase in friction, surface finishing of a rubber material, antislipping finishing, and connection by an antislipping wire, etc.

The intermediate part 16 is formed between the tip end part 15 and the root part 17 so that the tip end part 15 and the root part 17 can turn about respective axes of the two shafts 8a, 8b. That is, the intermediate part 16 comprises egg-shaped intermediate plates 9b, 12 mounted to the shafts 8a, 8b and formed with two holes, wire-guide pulleys 6e to 6h interposed between the intermediate plates 9b, 12, egg-shaped intermediate plates 11, 9a mounted likewise to the shafts 8a, 8b and formed with two holes, an intermediate plate 10 adjoining the intermediate plate 11 and formed on a side, through which the shaft 8b extends, with a disk-shaped projection 20, and guide pulleys 6a to 6d interposed between the intermediate plates 10, 9a (see FIG. 2).

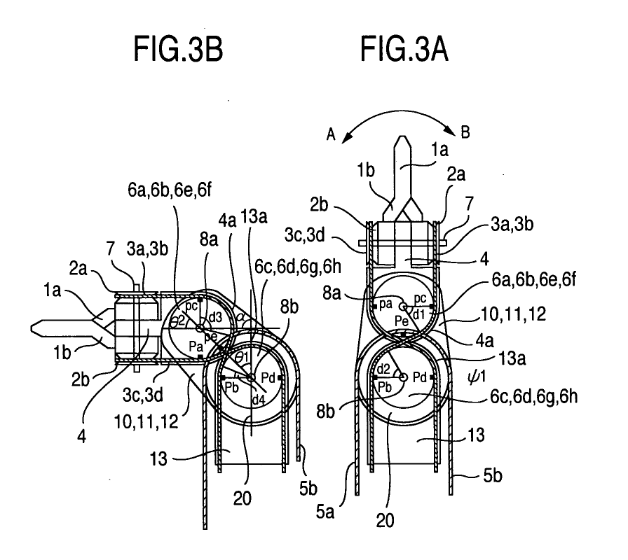

The intermediate plate 10 is formed to be low around the projection 20 and to make a portion around the hole, through which the shaft 8a extends, as high as the projection 20. The plate 10 and the plate 11 thus formed are joined together to form guide paths for the wires 5a, 5b (see FIG. 3). The rolling members 4b, 13b are interposed between the plates 11, 12. The shafts 8a, 8b extend through the holes formed on the plates 9a, 9b, the plates 10 to 12, and the pulleys 6a to 6g. The respective pulleys 6a to 6g are rotatable about the shafts 8a, 8b, and the rolling members 4b, 13b are brought into rolling contact with each other at the gear portions 4a, 13a. These members are preferably formed from a titanium alloy, which prevents generation of rust, etc. and is lightweight and high in stiffness.

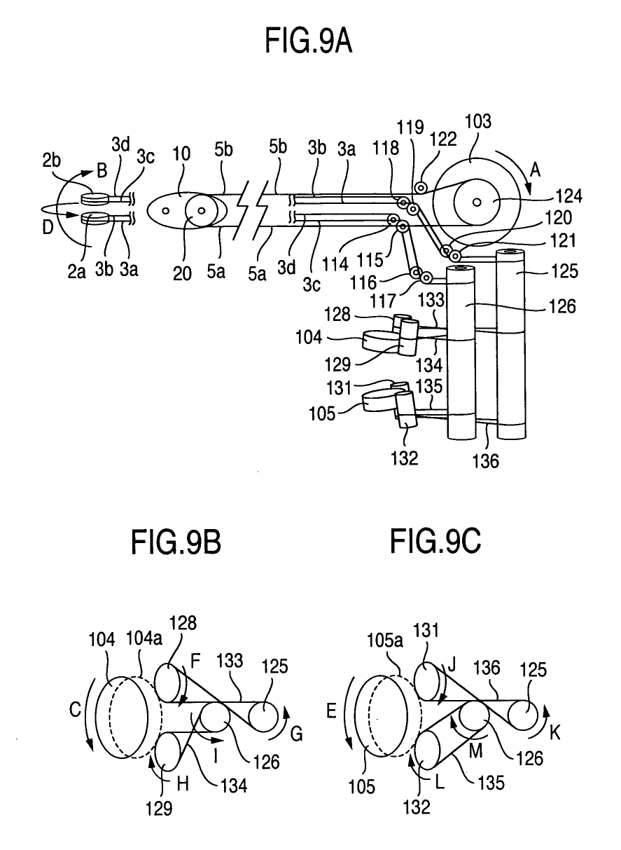

The wires 5a, 5b are fixed to an intersection Pe (see FIG. 2) of a circumference of the projection 20 on the intermediate plate 10 and a line connecting between the two shafts 8a, 8b, and mounted on an outer periphery of the projection 20. The wires 5a, 5b at tip end joint on a tip end of a surgical instrument pass through an interior of the cylinder portion 13 to be stretched around a vertical swing dial 103 of an operating part 102 on a hand side of the instrument as shown in FIG. 9. The wires 5a, 5b may comprise a length of continuous wire or two lengths of wire.

As shown in FIG. 4, the blade pulleys 2a, 2b are provided with grooves 22a, 22b, on which lengths of wire are stretched. The wire fixing portions 23a, 23b are mounted on parts of outer peripheries of the pulleys 2a, 2b. Parts of the wires 3a, 3b, 3c, 3d are fixed to the fixing portions 23a, 23b by means of bonding, welding, brazing, caulking, or the like.

The wire 3a fixed at one point to the blade pulley 2a is led to the pulley 6a, then to the pulley 6c, and fixed at one point to an outer periphery of a rotating shaft 125 of a hand grip 123 on the operating part 102 on a hand side as shown in FIG. 9 described later. Likewise, the wire 3b fixed at one point to the pulley 2a is fixed at one point to the outer periphery of the rotating shaft 125 of the hand grip 123. According to the embodiment, while the wire 3a and the wire 3b comprise a length of continuous wire, they may comprise two lengths of wire fixed to the blade pulleys 2a, 2b.

The wires 3c, 3d are mounted on a side of the blade 1b in the same manner as on a side of the blade 1a. That is, the wire 3c fixed at one point to the blade pulley 2b is led to the pulley 6b, then to the pulley 6d, and fixed at one point to an outer periphery of a rotating shaft 126 of the hand grip 123 on the operating part 102 on a hand side as shown in FIG. 9 described later. Likewise, the wire 3d fixed at one point to the pulley 2b is fixed at one point to the outer periphery of the rotating shaft 126 of the hand grip 123.

An operation of the tip end portion (a tip end joint or an instrument joint) of the surgical instrument, according to the invention, constructed in the above manner will be described below with reference to FIGS. 1 to 4. The gripper 14 rotates about the shaft 7 relative to the tip end part 15. On this occasion, when the shaft 7 is the same in a direction of rotation as the blades 1a, 1b, the gripper 14 is changed in orientation, and when the blades 1a, 1b rotate in a reverse direction to a direction, in which the shaft 7 rotates, the gripper 14 performs opening and closing actions. Specifically, when a first blade drive source, for example, a dial described later is manually operated to pull the wire 3b, the blade 1a moves in a closing direction. Conversely, when the wire 3a is pulled, the blade 1a moves in an opening direction. When a second blade drive source, for example, a dial described later is manually operated to pull the wire 3c, the blade 1b is closed, and when the wire 3d is pulled, the blade 1b is opened. When the wire 3a and the wire 3c are pulled together, or the wire 3b and the wire 3d are pulled together, the gripper 14 rotates about the shaft 7 to change a gripping direction. This is referred to as a swing action of the gripper joint.

As shown in FIG. 3, a swing angle α of the tip end part 15 is represented by the sum of an angle θ1 formed by the root part 17 and the intermediate part 16, and an angle θ2 formed by the intermediate part 16 and the tip end part 15. When the operating part on the hand side of the instrument is operated to rotate the vertical swing dial 103 shown in FIG. 9 described later to pull the wire 5a, the intermediate plate 10 is rotated about the shaft 8b in a A-direction shown in FIG. 3. At the same time, the shaft 8a, the tip end portion 4, the pulleys 6a, 6b, 6e, 6f, and the intermediate plates 9a, 9b are also rotated about the shaft 8b in the A-direction shown in FIG. 3. At this time, the gear portions 4a, 13a perform meshing actions while being brought into rolling contact with each other.

In the case where the gear portions 4a, 13a comprise gears having the same size, the tip end part 15 is rotated by θ1 about the shaft 8b and by θ2=θ1 about the shaft 8a when the intermediate plate 10 is rotated by θ1 about the shaft 8b. Thereby, an angle α, over which the tip end part 15 swings relative to the root part 17, becomes twice an angle, over which the intermediate plate 10 is rotated about the shaft 8b. When the operating part on the hand side of the instrument is operated to rotate the vertical swing dial 103 shown in FIG. 9 described later to pull the wire 5b, the tip end part 15 swings in a B-direction shown in FIG. 3. At this time, likewise swing in the A-direction, an angle α, over which the tip end part 15 swings relative to the root part 17, becomes twice an angle, over which the intermediate plate 10 is rotated about the shaft 8b.

In the case where a radius of the gear portion 4a is R times a radius of the gear portion 13a, the tip end part 15 is rotated by θ2=θ1/R about the shaft 8a when the intermediate plate 10 is rotated by θ1 about the shaft 8b. Accordingly, the tip end part 15 swings by an angle α=θ1(1+1/R) relative to the root part 17.

Central angles of those portions of the respective pulleys, with which the wires 3a, 3b are in contact, are varied according to a swing angle α. For example, the wire 3a comes into contact with the two pulleys 6a, 6c. The sum of central angles of those portions of the two pulleys 6a, 6c, with which the wire 3a comes into contact, is (d1+d2) in FIG. 3A and (d3+d4) in FIG. 3B. Since the gear portions 4a, 13a come into meshing contact with each other, the value is always constant and not dependent upon a swing angle α of the tip end joint, which is formed by the root part 17 and the tip end part 15. Accordingly, a path length of the wire between a point Pa and a point Pd and a path length of the wire between a point Pc and a point Pb are invariable irrespective of the angle α, and the wire is not changed in phase.

Here, the phase of the wire corresponds to an angle of opening and closing of the blades 1a, 1b and also corresponds to a position of the wire according to the angle of opening and closing, that is, a quantity, by which the drive pulls the wire. Since the wire is not changed in phase, the blades 1a, 1b do not open or close even when the joint of the tip end part 15 moves. Thereby, even when an angle α of the joint of the tip end part 15 is varied, no influences are produced on path length, phase and tension of those wires, which control the blades 1a, 1b provided further beyond the joint. Consequently, only a force applied on the moving part through the wires can be transmitted as a change in tension to a hand of an operator who operates the instrument joint (a tip end joint).

According to the embodiment, since only the wires 5a, 5b are operated at the time of swinging action to maintain the blades 1a, 1b constant in angle of opening and closing, it is unnecessary to adjust quantities, by which the wires 3a, 3b are pulled. Since the wire is not varied in path length even when a swing angle is changed, it is possible to prevent a situation, in which the wire is pulled to be unable to vary a swing angle. There is not generated a situation, in which the wire becomes loose when a swing angle is varied.

According to the embodiment, an operation of a wire can be correctly represented as an action of the instrument joint (a tip end joint) because of no interference on a wire, and an operation can be always performed with the same feeling of operation since the feeling of operation is not changed by that change in tension, which accompanies an operation. Since a force generated at the instrument joint can be transmitted as a change in tension to an operator, a situation of a therapeutic operation can be felt through an inner force sense and an operation is enabled while an operator feels an inner force sense as if treatment were normally performed directly by an operator's own hand, so that it is possible to grip an object with an appropriate force at a clinical site in the case where blood vessel and tissue is gripped and stitched. Thereby, a delicate treatment is enabled in low invasion.

Since a vertical swing wire (for example, the wire 5a, 5b) and a gripper operating wire (for example, the wires 3a to 3d) can be operated without any interference, an operation is made easy and an action of the instrument joint is made stable. It is possible to correctly perform the opening and closing action of the blades at any swing angle in a movable range and a swing action, which is performed while an object is gripped by the blades, and an operator can perform an operation in an intuitional feeling of operation without the need of any complicated operation. Since the intermediate part 16 having two centers of rotation is provided, it is possible to increase a range, in which the tip end part 15 can swing relative to the root part 17. Therefore, it is possible to treat the affected part, which is hidden behind internal organs.

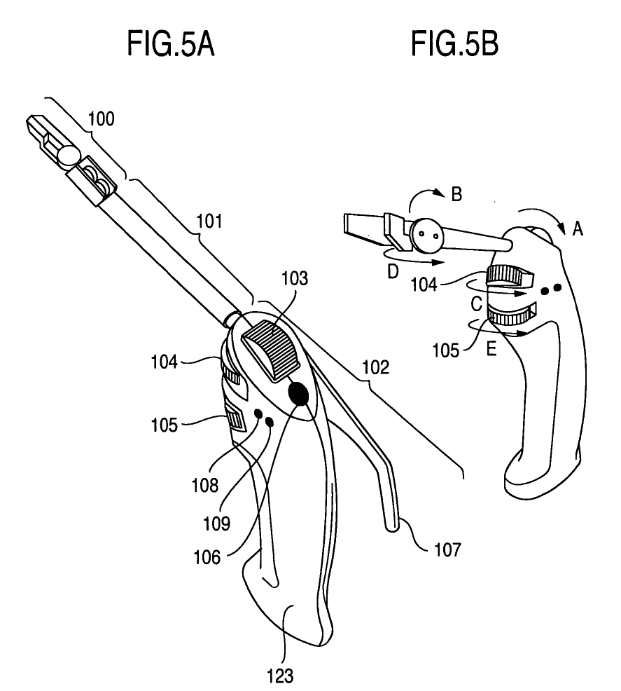

The construction of the gripper 14, the tip end part 15, the intermediate part 16, and the root part 17 described above presents an example of a fundamental construction adopted in the surgical instrument according to the invention. The fundamental construction shown in FIGS. 1 to 4 is also applied to a first modification of a surgical instrument according to the invention illustrated with reference to FIGS. 5 to 10.

Subsequently, features of the first modification according to the invention will be described in detail with reference to FIGS. 5 to 10.

In FIGS. 5 to 10, the reference numeral 100 denotes an instrument joint, 101 an arm part, 102 an operating part, 103 a vertical swing dial, 104 a lateral swing dial, 105 an opening and closing dial, 106 a lock button, 107 a stability holder, 108, 109 fixing parts, 110 a thumb, 111 a forefinger, 112, 113 fixing parts, 114 to 122 pulleys, 123 a hand grip, 124 a projection, 125, 126 rotating shafts, 127 an inclined mount surface, 128, 129, 131, 132 rotating shafts, 133 to 136 drive wires, 137, 138 dial mount angles, 139 a hand grip horizontal, 140 a hand grip vertical, 141 a hand grip surface, 142 a dial mount surface, 143 an interior angle of the stability holder, 144, 145 sides of the stability holder, 146 a shaft, 148, 149 holding plates, 150 a spring plate, 152 a slide hole, and 154, 155 mount surfaces.

The first modification of the surgical instrument (referred to as instrument) according to the invention comprises, as shown in FIG. 5, an instrument joint 100 including a gripper and a swing joint, an operating part 102 including a part, which an operator grasps to hold the same, a part, which operates swing (up and down and lateral) of an joint and opening and closing, and an arm part 101 including transmission means that couples the operating part 102 and the instrument joint 100 with each other to transmit an operation made with the operating part 102 to the instrument joint 100.

The operating part 102 comprises a dial 103 that operates vertical swing of a tip end joint (for example, the tip end joint acts in the B-direction when the dial is operated in the A-direction), a dial 104 that operates lateral swing of a gripper (the blades 1a, 1b) (for example, two blades 1a, 1b swing together in the same D-direction when the dial is operated in a C-direction), and a dial 105 that operates opening and closing of a gripper (blades) (for example, the blades 1a, 1b, respectively, act in an opening direction when the dial is operated in an E-direction, and perform reverse actions to those indicated by arrows in the figure when the dial is operated in a reverse direction). In addition, associated actions of the vertical swing dial 103, the lateral swing dial 104, and the opening and closing dial 105 in the operating part 102, and the instrument joint 100 will be described below in detail with reference to FIG. 9. The stability holder 107 in the operating part 102 serves to stably hold an instrument.

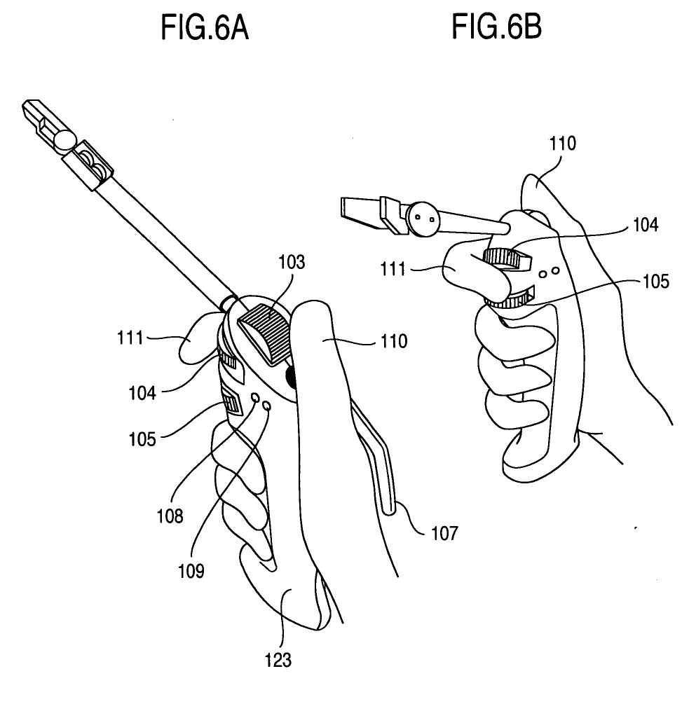

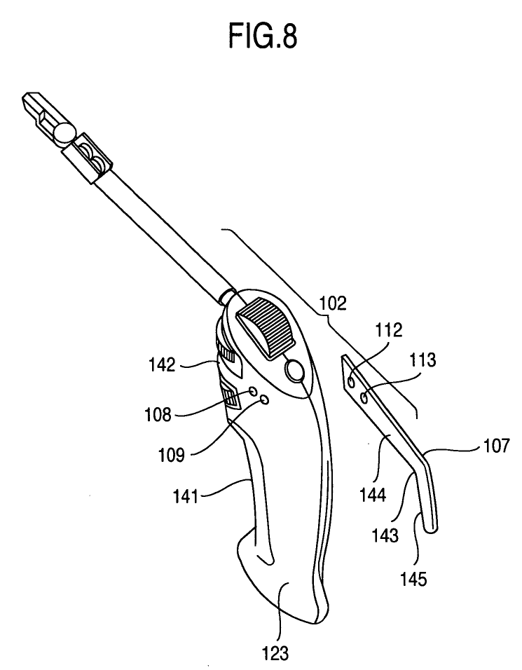

As shown in FIGS. 6 to 8, the first modification has a feature in a configuration and a construction, in which the hand grip 123 can be grasped by a palm, a middle finger, a ring finger, and a fifth finger. As can be appreciated from FIG. 8, the stability holder 107 is substantially L-shaped to comprise respective sides 144, 145 (an angle formed by the sides 144, 145 is not limited to 90 degrees (a L-shape) but suffices to be one for stable holding of an instrument, and normally forms an angle exceeding 90 degrees), and fixed to one side of the hand grip 123 by means of the fixing parts 112, 113 to stably hold an instrument. When an instrument is used with a right hand, the stability holder 107 is fixed to a right side of the hand grip 123 as shown in FIG. 8, and when an instrument is used with a left hand, the stability holder 107 is fixed to the fixing parts 108, 109 on a left side (details are described later in FIG. 8). In this manner, the stability holder 107 can be changed in a mount position depending upon the usage of an instrument, and serves to stably hold an instrument even in either case.

Described with reference to FIG. 6, when the hand grip 123 is grasped, the stability holder 107 is positioned between a thumb and a forefinger to have the side 145 in contact with a back of the hand and to have a palm brought into close contact with the hand grip 123. Further, since an interior angle 143 of the stability holder 107 is formed so that the sides 144, 145 of the stability holder and the hand grip 123 interpose therebetween a back of the hand and a palm lightly, the stability holder 107 is held between a thumb and a forefinger even when a middle finger, a ring finger, and a fifth finger separate from the hand grip, and since the hand grip 123 abuts against a palm, the instrument does not fall from a palm.

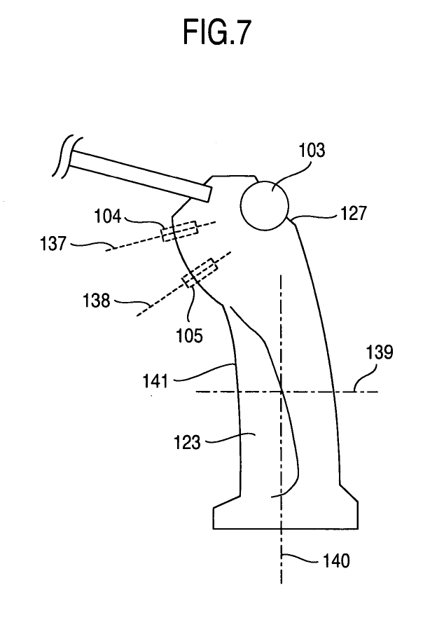

The both dials are arranged so that when the hand grip 123 is grasped, the lateral swing dial 104 and the opening and closing dial (a dial for opening and closing the blades 1a, 1b of the instrument joint 100) 105 are positioned in a position, in which a forefinger 111 is naturally extended. In FIG. 7, a plane (a plane perpendicular to the rotating shaft) of rotation of the lateral swing dial 104 is arranged to be made somewhat forwardly and downwardly of a hand grip horizontal 139 indicative of a horizontal direction of the hand grip 123. The opening and closing dial 105 arranged below the lateral swing dial 104 is arranged so that a plane 138 of rotation thereof is made further forwardly and downwardly of a plane 137 of rotation of the lateral swing dial. An angle between the plane 137 of rotation and the plane 138 of rotation is around 20 degrees, and a spacing therebetween amounts to a distance corresponding to one finger to one and a half finger (around 1 cm). Further, a dial mount surface 142, on which a dial is mounted, is shaped to project forwardly of a hand grip surface 141, with which a middle finger, a ring finger, and a fifth finger come into contact.

Since a finger skeleton is shaped so that when a forefinger 111 is swung vertically in a state, in which the hand grip 123 is grasped by a middle finger, a ring finger, and a fifth finger, the inner surface of a finger makes an arcuate movement about the root side joint of a finger, the lateral swing dial 104 and the opening and closing dial 105 are mounted as shown in FIG. 7 angularly relative to the planes of rotation thereof whereby either dial can be operated at a natural angle by a forefinger. As shown in FIG. 6, the lateral swing dial 104 and the opening and closing dial 105 are arranged above the hand grip 123 and in front (a lower portion (a projecting portion extended above the concave hand grip) of a connection of the arm part 101 and the operating part 102) of the operating part 102 so that a forefinger 111 can be operated without strain in a state, in which the hand grip 123 is grasped.

The vertical swing dial 103 is provided on an inclined mount surface 127, which comprises a surface inclined relative to a hand grip vertical line 140 (see FIG. 7) defining a vertical direction of the hand grip 123, so that when the hand grip 123 is grasped, it is liable to be operated by a thumb 110. With such construction, the vertical swing dial 103 can be operated in a posture, a thumb 110 is inclined forward (or obliquely forward), without strain. In other words, in a posture, in which a thumb 110 is inclined forward, that is, an angle between it and a forefinger 111 is small, a thumb 110 is wide in a movable range to be able to operate an object freely. According to the first embodiment, by providing the vertical swing dial 103 on the inclined mount surface 127 (not a vertical mount surface), it is possible to assume a posture of hand grip, in which the vertical swing dial 103 can be operated in a state, in which a wide movable range of a thumb 110 is ensured.

In FIG. 8, the fixing parts 108, 109 are ones, by which the stability holder 107 is fixed to the operating part 102. The stability holder 107 comprises parts 112, 113 to be fitted into the fixed parts 108, 109, and so is made detachable. Specifically, the fixed parts 108, 109 are concave in shape, and those parts of the stability holder 107, which are fitted into the fixed parts, are convex in shape. Alternatively, concave and convex may be reversed. Instead of simple concave and convex parts, convex parts may assume a shape of an elastic pawl in order to prevent play when being mounted. Further, a snap button may be used.

In case of grasp with a right hand, the stability holder 107 is mounted on a right side of the hand grip 123 as shown in FIGS. 5 and 6. In case of grasp with a left hand, the fixing parts 112, 113 are fitted into the fixed parts 108, 109 to be fixed to a left side of the hand grip 123 in a reverse manner to that in FIGS. 5 and 6. Thereby, it becomes possible to stably hold the instrument with either of lateral hands.

Subsequently, FIG. 9 is a view showing a construction, in which dials mounted on the hand grip and drive wires for driving of joints of the instrument joint are associated with each other. The drive wires around the dials are suspended and accommodated inside the operating part 102. Only parts related to drive and pulley portions of joints, which are connected together by wires, will be shown and connection thereof will be described below.

FIG. 9A shows a whole construction of a wire arrangement for dials and joints, FIG. 9B shows a wire arrangement from the lateral swing dial 104 to rotating shafts 126, 125, and FIG. 9C shows a wire arrangement from the opening and closing dial 105 to the rotating shafts 126, 125, the respective figures being viewed from above.

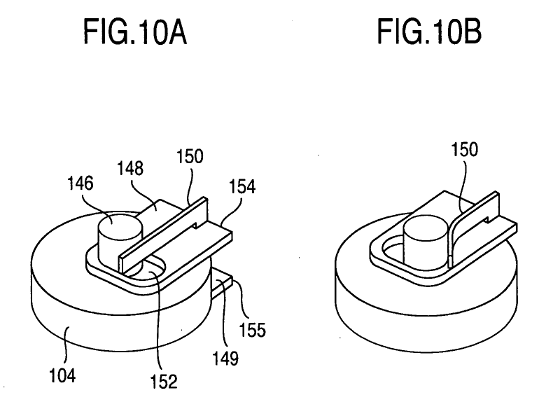

The lateral swing dial 104 and the opening and closing dial 105 are mounted to the hand grip 123 as shown in FIG. 10 to be able to slide. The rotating shafts 125, 126, 128, 129, 131, 132, respectively, are rotatably held on the hand grip 123. Pulleys 114, 115, 116, 117, 118, 119, 120, 121, 122 comprise parts that guide a wire, and, respectively, are rotatably mounted on the hand grip 123. The vertical swing dial 103 is shaped to have a projection 124 (a pulley may be fixed in place of a projection), and rotatably mounted on the hand grip 123.

The wires 3a, 3b, 3c, 3d, 5a, 5b are wired from the joints shown in FIG. 1, and wirings in the joints are illustrated in FIGS. 1 and 4. That is, as shown in FIG. 4, the blade pulley 2a is provided with the grooves 22a, in which the wires 3a, 3b are stretched, the wire fixing part 23a is mounted to a part of the outer periphery of the blade pulley 2a, and parts of the wires 3a, 3b are fixed to the fixing part 23a. The wires 3c, 3d are likewise fixed to a wire fixing part 24b of the blade pulley 2b. The wires 5a, 5b are fixed to the point Pe (see FIG. 2) on the circumference of the projection 20 on the intermediate plate 10 to be mounted on the outer periphery of the projection 20, and stretched around the projection 124 of the vertical swing dial 103 on the hand side of the instrument as shown in FIG. 9 described later.

The wire 3a fixed at one point to the blade pulley 2a is led to the pulley 6a, then to the pulley 6c (see FIG. 1), and finally fixed at one point to an outer periphery of the rotating shaft 125 (a pulley will do). Likewise, the wire 3b fixed at one point to the pulley 2a is led to the pulley 6f, then to the pulley 6h, and likewise fixed at one point to the outer periphery of the rotating shaft 125.

Described further in detail, the wires 5a, 5b are guided along paths by the pulley 122, wired along the projection 124 of the vertical swing dial 103, and led again as the wire 5b toward the joint. The wires 5a, 5b, respectively, are fixed to the projection 124 and connected to be able to transmit a drive force to the intermediate plate 10 according to rotation of the vertical swing dial 103, that is, the projection 124. The wires 5a, 5b may comprise a length of wire, or two lengths of wire.

The wire 3a is guided along a path by the pulleys 119, 121, led to the rotating shaft 125, wired along a predetermined groove (illustration of which is omitted) provided on the rotating shaft 125, guided as the wire 3b along a path by the pulleys 120, 118, and wired again toward the joint. The predetermined groove provided on the rotating shaft 125 may be simply concave in shape. The wire 3a and the wire 3b, respectively, are fixed to the rotating shaft 125 and connected to be able to transmit a drive force to the joint according to rotation of the rotating shaft 125. The wire 3a and the wire 3b may comprise a length of wire, or two separate lengths of wire.

The wire 3c is guided along a path by the pulleys 115, 117, led to the rotating shaft 126 (a pulley will do), wired along a predetermined groove (illustration of which is omitted) provided on the rotating shaft 126, guided as the wire 3d along a path by the pulleys 116, 114, and wired again toward the joint. The predetermined groove provided on the rotating shaft 126 may be simply concave in shape. The wire 3c and the wire 3d, respectively, are fixed to the rotating shaft 126 and connected to be able to transmit a drive force to the joints according to rotation of the rotating shaft 126. The wire 3c and the wire 3d may comprise a length of wire, or two separate lengths of wire.

The wire 133 is one wired to transmit a drive force between the rotating shaft 125 described above and the rotating shaft 128, and fixed at one point to the rotating shaft 128 and at one point to the rotating shaft 125. The wire 133 is wired as shown in FIG. 9B in a path to intersect between the rotating shaft 128 and the rotating shaft 125. At this time, wiring is made along the predetermined grooves of the rotating shafts 128, 125 to be varied in level so that the wire 133 does not come into contact with each other. The wire 134 is one wired to transmit a drive force between the rotating shaft 126 described above and the rotating shaft 129, and fixed at one point to the rotating shaft 129 and at one point to the rotating shaft 126. The wire 134 is wired as shown in FIG. 9B in a path to intersect between the rotating shaft 129 and the rotating shaft 126. At this time, wiring is made along the predetermined grooves of the rotating shafts 129, 126 to be varied in level so that the wire 134 does not come into contact with each other.

The wire 135 is one wired to transmit a drive force between the rotating shaft 126 described above and the rotating shaft 132, and fixed at one point to the rotating shaft 132 and at one point to the rotating shaft 126. At this time, the wire 135 is wired along the predetermined grooves of the rotating shafts 132, 126. Further, the wire 136 is one wired to transmit a drive force between the rotating shaft 125 described above and the rotating shaft 132, and fixed at one point to the rotating shaft 131 and at one point to the rotating shaft 125. The wire 136 is wired as shown in FIG. 9C in a path to intersect between the rotating shaft 131 and the rotating shaft 125. At this time, wiring is made along the predetermined grooves of the rotating shafts 131, 125 to be varied in level so that the wire does not come into contact with each other. In addition, all the predetermined grooves may be simply concave in shape. In that region in the intermediate part 101, which is free of contact with the pulleys, the wires 3a, 3b, 3c, 3d, 5a, 5b can be replaced by rods. The rods are made of a material, such as metal, FRP, etc., of less elongation, the respective wires wired from the pulleys, respectively, are fixed to the rods, and the rods having predetermined lengths are again fixed at one ends thereof to the wires. The rods are set in length so that they do not interfere with a construction such as pulleys, etc. when operating in a whole operating region of the wires. By replacing parts of the wires by rods of high strength, it is possible to expect an effect that the drive force transmitting paths are enhanced in strength and influences of elongation of the wires are suppressed.

Subsequently, description will be given to actions of the dials (the dials for vertical swing, lateral swing, and opening and closing) and the drive wires. When the vertical swing dial 103 is operated by a thumb 110 in the A-direction, the wire 5b is pulled, and the intermediate plate 10 is swung about a center of rotation of the projection 20 in the B-direction. When the vertical swing dial 103 is operated in a reverse direction to the A-direction, the wire 5a is pulled, and the intermediate plate is swung in a reverse direction to the B-direction. At this time, an operation is made so that a direction, in which the vertical swing dial 103 is operated by a finger, and a direction, in which the joint swings, itself are consistent with each other.

When the lateral swing dial 104 is to be operated, a forefinger 111 pushes the dial 104 to rotate the same (a detailed construction for the pushing rotation will be described with reference to FIG. 10 described later). Applied between the dial 104 and the rotating shafts 128, 129 is a configuration that enables transmission of torque. For example, the configuration enables transmission of torque by frictional forces, or transmission by a gear configuration. When a forefinger 111 is separated from the dial 104, the dial 104 is separated from the rotating shafts 128, 129 to bring about a state, in which torque cannot be transmitted mutually.

When the lateral swing dial 104 is operated in the C-direction (see FIG. 9B), the rotating shafts 128, 129 are rotated in a F-direction and a H-direction, respectively. Then, the rotating shafts 125, 126 are rotated through the wires 133, 134 in a G-direction and a I-direction. Thereby, the wires 3a, 3c are pulled, and the pulleys 2a, 2b in the grippers swing together in the D-direction. When the lateral swing dial 104 is operated in a reverse direction to the C-direction, torque is transmitted in reverse rotation to that described above, and the pulleys 2a, 2b in the grippers swing together in a reverse direction to the D-direction. At this time, an operation is made so that a direction, in which the lateral swing dial 104 is operated by a forefinger 111, and a direction, in which the joint swings, itself are consistent with each other.

When the opening and closing dial 105 is to be operated, a forefinger 111 pushes the dial 105 to rotate the same. Applied between the dial 105 and the rotating shafts 131, 132 is a configuration that enables transmission of torque. For example, the configuration enables transmission of torque by frictional forces, or transmission by a gear configuration. When a forefinger 111 is separated from the dial 105, the dial 105 is separated from the rotating shafts 131, 132 to bring about a state, in which torque cannot be transmitted mutually.

When the opening and closing dial 105 is operated in the E-direction (see FIG. 9C), the rotating shafts 131, 132 are rotated in a J-direction and a L-direction, respectively. Then, the rotating shafts 125, 126 are rotated through the wires 135, 136 in a K-direction and a M-direction. Thereby, the wires 3a, 3d are pulled, and the pulley 2a in the gripper swings in the D-direction and the pulley 2b swings in a reverse direction to the D-direction, so that the grippers (the blades 1a, 1b) are opened. When the opening and closing dial 105 is operated in a reverse direction to the E-direction, torque is transmitted in a reverse rotation to that described above, the pulley 2a in the gripper rotates in a reverse direction to the D-direction, and the pulley 2b rotates in the D-direction, so that the gripper is closed.

Here, when the lateral swing dial 104 is operated in the C-direction, this operation is accompanied by rotation of the rotating shaft 131 through the rotating shaft 125 in the J-direction and rotation of the rotating shaft 132 through the rotating shaft 126 in a reverse direction to the L-direction, but the rotating shafts 131, 132 are put in a state, in which torque is not transmitted between them and the opening and closing dial 105, so that any interference is not caused. Likewise, when the opening and closing dial 105 is operated, any interference with the lateral swing dial 104 is not caused.

As described above, while the joint construction according to the embodiment comprises respective drive wires provided on respective blades and made individually operable in order to realize a multiple degree-of-freedom construction having a high operability, connection of an operating part and joints is made according to the embodiment whereby relatively different operations of two blades, in which the two blades are moved simultaneously in the same direction, or the two blades are moved simultaneously in opposite directions, can be allotted to independent dials, respectively.

Therefore, when the joint construction according to the embodiment is to be operated manually, operation of one dial can realize a swinging action, in which the two blades are operated together in the same direction, an opening and closing action, in which the two blades are operated in different directions, and an operation of the multiple degree-of-freedom joint intuitionally and easily unlike a complicated operating method, in which two wires are allotted to separate operating means and an operator operates the separate operating means by the same amounts simultaneously in the same direction, or operates the means simultaneously in different directions. Operation of the joint is increased in freedom and operability is enhanced whereby a further complicate treatment can be performed further safely. Since an operation can be performed not electrically but directly and manually, an operator's hand can feel a grasping force by the instrument at a tip end, and sense at the time of contact with an organ, and a delicate state of an applied force can be controlled making use of a touch, which cannot be obtained with an electrically controlled equipment.

FIG. 10 shows a slide operating mechanism for the lateral swing dial 104. Since the opening and closing dial 105 is the same in construction as the lateral swing dial 104, it is unified and described in an explanation of the lateral swing dial 104 (referred below to as dial 104). FIG. 10A shows a state, in which the dial 104 is not pushed, and FIG. 10B shows a state, in which the dial is pushed.

The dial 104 is formed with the projection 146, which becomes a rotating shaft. Although not shown in FIG. 10, a similar projection is provided in a corresponding position on the opposite side. The projection is referred as a rotating shaft projection 147 (not shown) for the sake of convenience. The holding plate 148 is provided with the slide hole 152, through which the rotating shaft 146 extends, and comprises a part having the function of making the rotating shaft 146 extending therethrough to hold the dial 104. The holding plate 149 having the same shape as that of the holding plate 148 is mounted in a corresponding position with respect to the holding plate 148 to make the rotating shaft 146 extending therethrough to interpose the dial 104 between it and the holding plate 148. The holding plates 148, 149 are fixed at the mount surfaces 154, 155 to the hand grip 123. The spring plate 150 is made of an elastic body, and the spring plate is fixed at one end thereof to the holding plate 148 and has the other end thereof pushing the rotating shaft 146 along the groove of the slide hole 152 in one direction. The spring plate 151 (not shown) made of an elastic body like the spring plate 150 is provided on the holding plate 149 in a symmetrical position with respect to the spring plate 150 to push the rotating shaft 147 in one direction in the same manner as the rotating shaft 146.

A longitudinal direction of the slide hole 152 on the holding plate 148 corresponds to a direction, in which the dial 104 slides. When the dial 104 is to be operated, the dial 104 is pushed against an end of the slide hole 152 on an opposite side along the slide hole 152 by a force of a forefinger as shown in FIG. 10B, so that the spring plate 150 (and the spring plate 151) is bent as shown in FIG. 10B. In a state shown in FIG. 10B, the dial 104 is put in a state, in which it comes into contact with the rotating shafts 128, 129 shown in FIG. 9 to transmit rotation of the dial 104 to the rotating shafts 128, 129, and when a forceps (the blades 1a, 1b) is to be operated, it suffices to rotate the dial 104 in a state shown in FIG. 10B. In the state shown in FIG. 10B, the spring plate 150 (and the spring plate 151) generates a force for restoration of a state shown in FIG. 10A, so that the state shown in FIG. 10A is restored upon separation of a forefinger 11. In the state shown in FIG. 10A, the dial 104 is separated from the rotating shafts 128, 129, so that rotation cannot be transmitted therebetween.

While the construction, function and action of the surgical instrument according to the second embodiment of the invention have been described, the following features are specifically provided by the second embodiment. That is, in a fundamental configuration, in which the hand grip is grasped by a palm and the stability holder has a back of the hand in holding the instrument, an operation by a thumb with a thumb obliquely forward or upward enables the shoulder joint (the armpit) to be clamped to the body, so that a stable posture for surgery can be ensured and fatigue is hard to generate. Thereby, it is possible to operate the instrument for a long time.

It is possible to hold the hand grip by a middle finger, a ring finger, a fifth finger, and a palm and to operate the lateral swing dial and the opening and closing dial in a natural posture (without strain) of a forefinger. In a specific example, a relative distance of the both dials is made around 1 cm and a relative angle (an angle formed by the plane 137 and the plane 138 in FIG. 7) of the both dials is made around 20 degrees. Thereby, a forefinger makes it possible to make actions of lateral swing and opening and closing without strain and without fail.

The fundamental function and operation of the first modification, to which the construction shown in FIGS. 1 to 4 is applied, widens a range, in which a forceps can swing, and enables ensuring multiple degrees of freedom, so that it is possible to expect an improvement in operability. Since the tip end joint is constructed such that a swinging action of a forceps is not accompanied by forward movements of a forceps, the tip end of the instrument is not shifted much by such swing and a delicate and further exact, medical operation can be realized while a swinging operation is added in a flow of the medical operation. Further, no strain is imposed on a posture during the operation and complicated movements of the tip end of the instrument can be made with ease, so that the operation of the instrument with less fatigue and with constant stability is enabled.

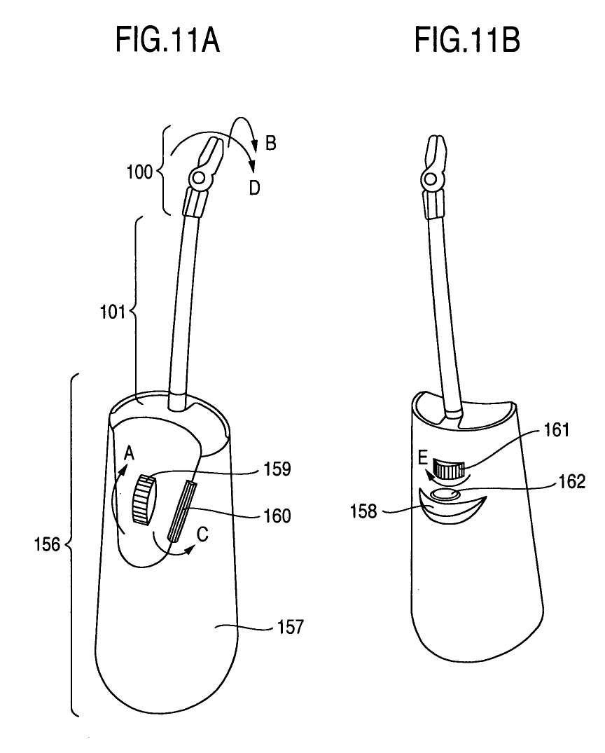

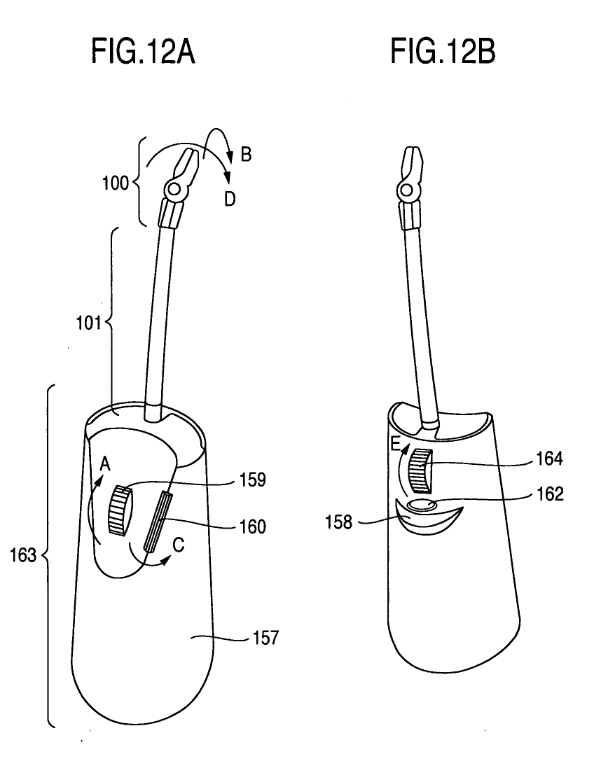

Subsequently, an explanation will be given to a second modification with reference to FIG. 11. In addition, since a third modification of the invention shown in FIG. 12 is different in the construction of an opening and closing dial from the second modification but is common thereto in many constituent elements, respective constituent elements in the second and third modifications are described here.

In FIGS. 11 to 18, the reference numeral 156 denotes an operating part, 157 a hand grip, 158 a finger rest (a stopper for movement of a middle finger), 159 a vertical swing dial, 160 a lateral swing dial, 161 an opening and closing dial, 162 a lock button, 163 an operating part, 164 an opening and closing dial, 165 an operating part, 167 a wrist, 168 a thumb, 169 a forefinger, 170 a middle finger, 171 a ring finger, 172 a fifth finger, 173 a mount surface, 174 an operating part axis, 175 a dial rotating shaft, 176 a pulley, 177 a pulley, 178 a pulley, 179 a pulley, 180 a pulley, 181 a pulley, 182 a projection, 183 a rotating shaft, 184 a rotating shaft, 185 a rotating shaft, 186 a rotating shaft, 187 a rotating shaft, 188 a rotating shaft, 189 a drive wire, 190 a drive wire, 191 a drive wire, 192 a drive wire, 193 a rotating shaft, and 194 a rotating shaft.

The second modification comprises the instrument joint 100 including a gripper and a swing joint, an operating part 156 including a part, which an operator grasps in order to hold the same, a part, which operates swing of an joint and opening and closing, and an arm part 101 including transmission means that couples the operating part 156 and the instrument joint 100 with each other to transmit an operation made with the operating part 156 to the instrument joint 100. FIGS. 11A and 11B are views showing outward appearances of a front surface and a back surface of the second modification.

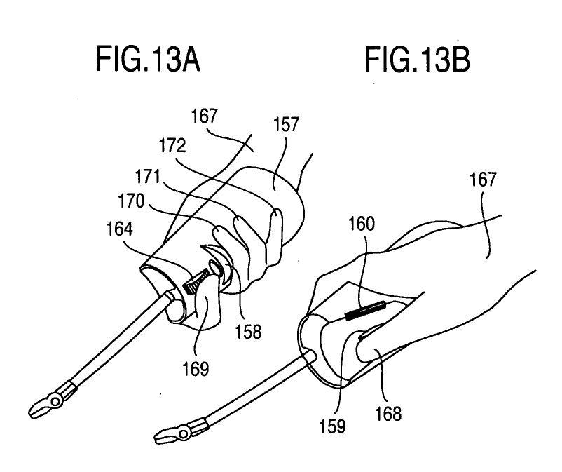

The reference numeral 157 denotes a hand grip, which an operator grasps, and in which an operating force transmission means coupling a dial operating system and the instrument joint 100 with each other is provided. The reference numeral 158 denotes a finger rest formed convexly on the hand grip 157, the finger rest permitting a finger to be put thereon when the opening and closing dial 161 is operated, and serving to prevent other fingers from touching the dial (details will be described with reference to FIG. 13). The reference numeral 159 denotes a vertical swing dial for operation of vertical swing of the instrument joint 100, and when the vertical swing dial is operated in the A-direction, the instrument joint (tip end joint) 100 acts.

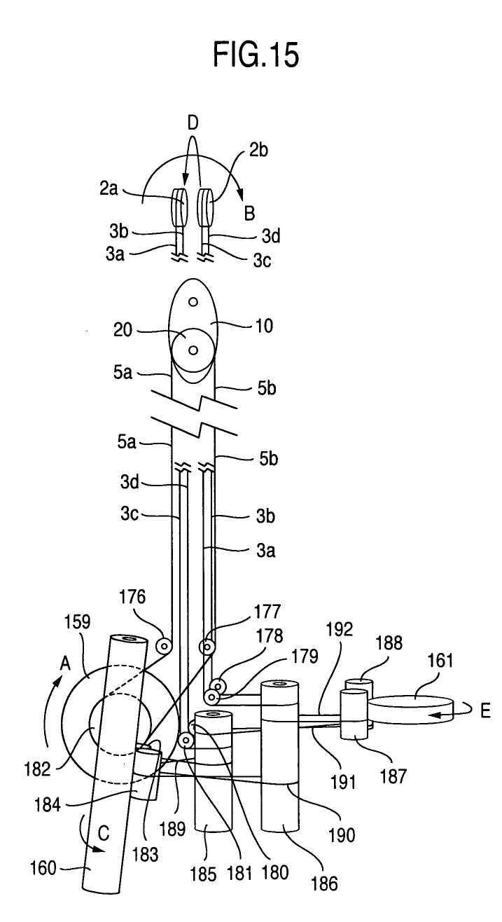

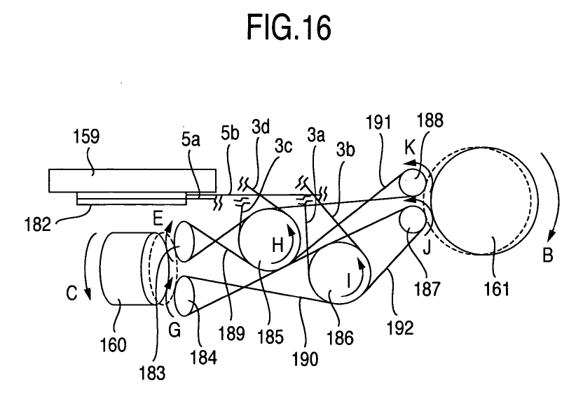

The reference numeral 160 denotes a lateral swing dial for operation of lateral swing of the hand grip, and when the dial 160 is operated in the C-direction, the two blades 1a, 1b (see FIG. 3) swing together in the same D-direction. The reference numeral 161 denotes an opening and closing dial for operation of opening and closing of the gripper, and when the dial 161 is operated in the E-direction, the blades 1a, 1b, respectively, act in an opening direction. When the dial is operated in a reverse direction, it performs a reverse action to that indicated by arrows in FIG. 11B to act in a closing direction. Details of these actions will be described with reference to FIGS. 15 and 16. The reference numeral 162 denotes a lock button for the opening and closing dial 161.

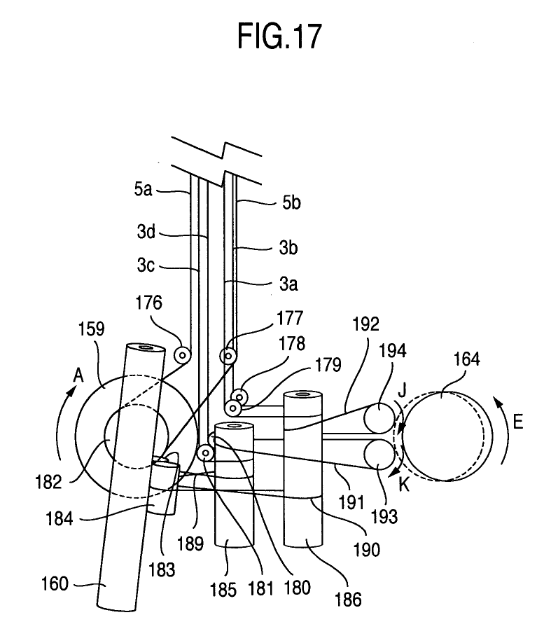

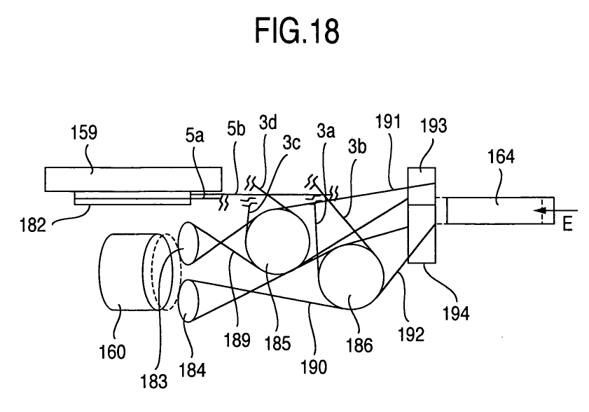

FIG. 12 shows a third modification of the invention. The third modification is different from the second modification in construction and operation such that orientation, in which the opening and closing dial 164 is operated, is different therebetween. An internal construction of the instrument will be described with reference to FIGS. 17 and 18.

FIG. 13 is a view showing a state, in which the operating part 163 in the third modification of the invention is grasped by a palm and fingers. In addition, FIG. 13 is naturally applied to the operating part in the second modification, and so an explanation is given taking the third modification as an example. As compared with the first modification, one of features of the second and third modifications resides in that the vertical swing dial and the opening and closing dial are operated by a thumb 168, and at that time, the vertical swing dial 159 and the lateral swing dial 160 are made different in direction of rotation, that is, 90 degrees in the A-direction and in the C-direction, to thereby prevent erroneous operation of the both dials. A further feature resides in a difference in the external form of the operating part and a way, in which the arm part is mounted (while the operating part in the first modification is in the form of a pistol, the operating parts in the second and third modifications are in the form of an oiler as shown in FIG. 11).

In FIG. 13, the hand grip 157 is grasped by a middle finger 170, a ring finger 171, a fifth finger 172, and a palm. The vertical swing dial 159 and the lateral swing dial 160 are operated by a thumb 168, and the opening and closing dial 164 (the opening and closing dial 161 in the second modification) is operated by a forefinger 169.

With the construction shown in FIG. 13, it is possible to get a touch to the operation dials 159, 160 in a state, in which a thumb assumes a natural (without application of a surplus force) posture relative to a wrist. Since a thumb can move in a wide range in such state, a large operation is enabled with ease. In such state, movements of the vertical swing dial 159, for which a first joint of a thumb is used, can be easily made in the direction of operation. Since a thumb can move in a wide range, not only the vertical swing dial 159 but also the lateral swing dial 160 arranged laterally thereof can also be operated with the same thumb.

When a thumb should be moved to the lateral swing dial 160 from the vertical swing dial 159, a thumb is moved transversely, which movement is made in a direction, in which a thumb can move easily. Since a direction (see the C-direction in FIG. 12), in which the lateral swing dial 160 is operated, is consistent with such transverse direction, the lateral swing dial 160 can be operated in a natural movement when it is operated, so that operability is not damaged.

Further, since it is possible to grasp the hand grip 157 and operate the instrument in a natural state, in which a wrist is extended, grasp and operation become easy to enable a decrease in fatigue. Since grasp and operation are enabled in an easy posture, sense of a tip of a finger can be accurately reflected on the operation of the tip end of the instrument with the result that it is possible to realize a delicate operation of the instrument. Since the instrument can be operated delicately, it is possible to improve an operation of medical treatment in safety and accuracy.

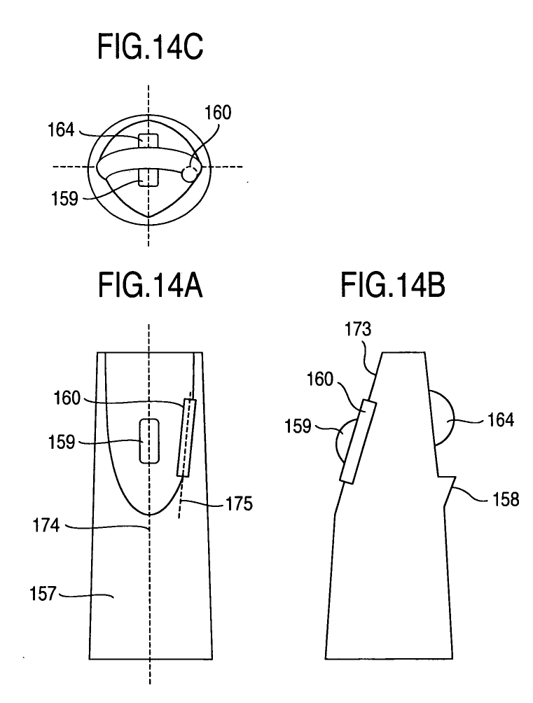

FIG. 14 is a view showing a configuration of the operating part 163 in the third modification of the invention. In addition, FIG. 14 can be naturally applied to the operating part in the second modification, and so an explanation is given taking the third modification as an example.

By forming the hand grip 157 in the operating part to make the same substantially conical or substantially tapered form cross section of which substantially elliptical, the hand grip 157 can be easily received and carried in a palm. Further, since the hand grip is larger on a side toward a fifth finger in cross sectional area than on a side toward a middle finger, it is possible to increase a force, with which the hand grip is grasped by a ring finger and a fifth finger, thus enabling grasping the hand grip further firmly. Further, since the mount surface 173 for the vertical swing dial 159 and the lateral swing dial 160 is defined by an inclined surface, which is directed inward from the substantially conical configuration of the hand grip 157, a thumb can touch the dials 159, 160 in a posture, in which it can move in a wide range (angle), in a state of grasp as shown in FIG. 13B, thus enabling improving operability by a thumb. In an example shown, the mount surface 173 for the dials 159, 160 is concave and tapered in shape, the dial 159 is arranged centrally of the recess, and the dial 160 is arranged at a right end portion (a left end portion will do) of the recess.

As shown in FIG. 14B, the finger rest 158 projects from the substantially conical configuration of the hand grip 157, and as shown in FIG. 13A, the hand grip is grasped in a position, in which a middle finger 170 touches the finger rest 158, whereby the hand grip can be always grasped in the same position and the positional relationship can be made the same whenever a thumb 168 and a forefinger 169 are operated. Since the finger rest 158 limits a range, in which a middle finger 170 moves, to prevent a middle finger from touching the opening and closing dial 164, it is possible to prevent an erroneous operation, in which a middle finger 170 touches the opening and closing dial 164 while the instrument is grasped.

FIG. 15 is a view showing a state, in which drive wires are stretched between an joint mechanism at the tip end and an operating mechanism on the hand side in the second modification of the invention. FIG. 16 is a view showing a situation of wiring around the operating dials shown in FIG. 15 as viewed from above.

With reference to FIG. 15 showing an operating force transmission mechanism for the operating part 156 and wiring and FIG. 16 showing a situation of wiring between the operating dials 159, 160, 161 and the rotating shafts 185, 186, to which wires led from the instrument joint 100 are fixed, an explanation will be given to transmission of an operating force and a situation of wiring.

The pulleys 176, 177, 178, 179, 180, 181 and the rotating shafts 183, 184, 185, 186, 187, 188 are rotatably provided within the hand grip 157. The projection 182 is provided around the rotating shaft on the vertical swing dial 159. The lateral swing dial 160 and the opening and closing dial 161 have the same structure as those in FIG. 10 and are provided on the hand grip 157.

When the lateral swing dial 160 is slid to the inner part by a finger, it comes into contact with the rotating shafts 183, 184 at the same time, so that torque can be transmitted mutually. Upon separation of a finger, the lateral swing dial is pushed back by forces of the spring plates 150, 151 in the same manner as that shown in FIG. 10, there comes out a state, in which torque is not transmitted between the lateral swing dial 160 and the rotating shafts 183, 184. Likewise the lateral swing dial 160, when the opening and closing dial 161 is slid to the inner part by a finger, it comes into contact with the rotating shafts 187, 188 at the same time, so that torque can be transmitted mutually. Upon separation of a finger, the opening and closing dial is pushed back by forces of the spring plates 150, 151 in the same manner as that shown in FIG. 10, there comes out a state, in which torque is not transmitted between the opening and closing dial and the rotating shafts 187, 188.

The drive wire 189 is wired between the rotating shaft 183 and the rotating shaft 185 to enable transmission of torque. The drive wire 190 is wired between the rotating shaft 184 and the rotating shaft 186 to enable transmission of torque. Further, the drive wire 192 is wired between the rotating shaft 187 and the rotating shaft 186 to enable transmission of torque. The drive wire 191 is wired between the rotating shaft 188 and the rotating shaft 185 to enable transmission of torque.

Here, drive wires extending from the instrument joint (tip end joint) 100 comprise the six drive wires 3a, 3b, 3c, 3d, 5a, 5d as shown in FIG. 1. The wire 3a is led to the rotating shaft 186 by the pulley 179 to be fixed in a predetermined position. The wire 3b is led to the rotating shaft 186 by the pulley 178 to be fixed in a predetermined position. Tension is transmitted to the wires 3a, 3b according to rotation of the rotating shaft 186 to transmit a rotating angle of the rotating shaft 186 to a rotating angle of the blade pulley 2a at the tip end joint. The wire 3c is led to the rotating shaft 185 by the pulley 181 to be fixed in a predetermined position. The wire 3d is led to the rotating shaft 185 by the pulley 180 to be fixed in a predetermined position. Tension is transmitted to the wires 3c, 3d according to rotation of the rotating shaft 185 to transmit a rotating angle of the rotating shaft 185 to a rotating angle of the blade pulley 2b at the tip end joint.

The wire 5a is led to the projection 182, provided on the vertical swing dial 159, by the pulley 176 to be fixed at one point on the projection 182. The wire 5b is led to the projection 182, provided on the vertical swing dial 159, by the pulley 177 to be fixed at one point on the projection 182. The wires 5a, 5b may comprise a length of wire. Rotation of the vertical swing dial 159 gives tension to the wires 5a, 5b to enable transmitting a rotating angle of the vertical swing dial 159 to the projection 20 provided on the intermediate plate 10.

In specific actions, for example, when the vertical swing dial 159 is rotated in the A-direction, the projection 20 rotates in synchronism with the vertical swing dial 159 and the instrument joint 100 rotates in the B-direction. When the lateral swing dial 160 is pushed to bring into a state, in which torque can be transmitted between it and the rotating shafts 183, 184, and when the lateral swing dial 160 is rotated in the C-direction, the rotating shaft 183 is rotated in the F-direction and the rotating shaft 184 is rotated in the G-direction (see FIG. 16), and further, the rotating shaft 185 is rotated through the drive wire 189 in the H-direction and the rotating shaft 186 is rotated through the drive wire 190 in the I-direction. When the rotating shaft 185 is rotated in the H-direction and the rotating shaft 186 is rotated in the I-direction, the wires 3a, 3c are pulled and the blade pulleys 2a, 2b swing together in the D-direction.

At this time, while rotations of the rotating shafts 185, 186 are also transmitted to the rotating shafts 188, 187, the rotating shafts 188, 187 only idle unless the opening and closing dial 161 is pushed, so that torque is not transmitted to the opening and closing dial 161. Since the rotating shafts 188, 187, respectively, are about to rotate in a sense, in which the opening and closing dial 161 is rotated in a reverse direction, rotations of the rotating shafts 188, 187 interfere with each other to be fixed when the opening and closing dial 161 is pushed. Thereby, movements of the rotating shafts 185, 186 can be fixed simultaneously with the result that it is possible to fix the lateral swing dial 160 in movement to fix the swinging motion.

When the opening and closing dial 161 is rotated in the E-direction in a state, in which the opening and closing dial 161 is pushed to enable transmission of torque between it and the rotating shafts 188, 187, the rotating shaft 187 rotates in the J-direction and the rotating shaft 188 rotates in the K-direction. Further, the rotating shaft 185 is rotated through the drive wire 191 in an opposite direction to the H-direction and the rotating shaft 186 is rotated through the drive wire 192 in the I-direction, whereby the wires 3a, 3d are pulled, so that the blade pulley 2a rotates in the D-direction, and the blade pulley 2b rotates in an opposite direction to the D-direction. Thereby, the blades 1a, 1b are rotated in a direction, in which they open relative to each other. When the opening and closing dial 161 is rotated in an opposite direction to the E-direction, the blades 1a, 1b are rotated in a direction, in which they close relative to each other.

At this time, while rotations of the rotating shafts 185, 186 are also transmitted to the rotating shafts 183, 184, the rotating shafts 183, 184 only idle unless the lateral swing dial 160 is pushed, so that torque is not transmitted to the lateral swing dial 160. At this time, since the rotating shafts 183, 184, respectively, are about to rotate in a sense, in which the lateral swing dial 160 is rotated in opposite directions, rotations of the rotating shafts 183, 184 interfere with each other to be fixed when the lateral swing dial 160 is pushed. Thereby, movements of the rotating shafts 185, 186 can be fixed simultaneously with the result that it is possible to fix the opening and closing dial 161 in movement to fix the opening and closing motion.

In this manner, swinging operations, allotted to the respective dials, in predetermined directions, and opening and closing operations of the gripper can be performed independently by using the vertical swing dial 159, the lateral swing dial 160, and the opening and closing dial 161 and operating the respective dials to perform operations, in which the drive wires connected to the instrument joint 100 are appropriately put in cooperation with one another. Thereby, an operator can easily realize an intended action only through movements of a tip of a finger without any complicated operation.

FIG. 17 is a view showing a state, in which drive wires are stretched between an joint mechanism at the tip end and an operating mechanism on the hand side in a third modification of the invention. FIG. 18 is a view showing a situation of wiring around the operating dials shown in FIG. 17 as observed from above. FIGS. 17 and 18 are views showing the third modification, and the views, respectively, are substantially the same as FIGS. 15 and 16, which show the second modification. However, the third modification is different from the second modification in orientation of the opening and closing dial 164 relative to the opening and closing dial 161 in FIG. 15 and orientations of the rotating shafts 194, 193 resulted from such difference. Since the drive wires 191, 192 between the rotating shafts 185, 186 and the rotating shafts 193, 194 comprise a length of wire, torque can be transmitted even when orientations of the rotating shafts 193, 194 are deviated 90 degrees from those in FIG. 15. The remaining construction is the same as that in FIGS. 15 and 16. When the opening and closing dial 164 is rotated in the E-direction, the blades 1a, 1b act in opening directions, and when the opening and closing dial 164 is rotated in an opposite direction to the E-direction, the blades 1a, 1b turn in closing directions.

In this manner, swinging operations, allotted to the respective dials, in predetermined directions, and opening and closing operations of the gripper can be performed independently by using the vertical swing dial 159, the lateral swing dial 160, and the opening and closing dial 161 and operating the respective dials to perform operations, in which the drive wires connected to the instrument joint 100 are appropriately put in cooperation with one another. Thereby, an operator can easily realize an intended action only through movements of a tip of a finger without any complicated operation.

While the fundamental function and operation of the embodiment of the invention and the first, second, and third modifications thereof have been described, the invention can fulfill the following function and operation especially. That is, for the operability of the surgical instrument, the tip end of the instrument (forceps) is movable in a wide range and multiple in degree of freedom, an operation in multiple degrees of freedom can be realized by movements of a thumb and a forefinger without strain, and a forceps can be operated in an easy posture. Since it is possible to bend the tip end in multiple degrees of freedom even in an easy posture of operation to make an approach to the affected part in a desired posture of the tip end, it is unnecessary to use a whole arm to perform an operation. Therefore, fatigue is not generated since an operation can be performed in an easy posture. Further, since an operation can be performed in a posture, in which the armpit is clamped, while holding the instrument in a grasping posture with no burden on a wrist, a delicate operation is enabled to result in an increase in safety.

Since the tip end can be operated in multiple degrees of freedom, it is possible to heighten a posture of the instrument at the tip end, which approaches the affected part, in freedom.

Since the tip end joint can be operated in posture only by movements of a tip of a finger, an operation to a desired posture of the tip end is enabled without interruption of the operation of medical treatment. Since the operation of medical treatment is not interrupted, it is possible to shorten time for medical treatment.

A direction of swing of a forceps and opening and closing actions, respectively, are allotted to one operating dial, operation of the operating dial in cooperation with the drive wires for operation of the joint is made without any complicated operation, and the tip end joint can be operated easily, so that an operation can be made further intuitionally and desirably to achieve an increase in operability and safety.

Further, a forceps can be moved in a large range owing to the construction, in which the joints, respectively, do not interfere in freedom with each other. Further, since a state of large swing does not affect subsequent operability and operating forces in freedom, it is possible to maintain a predetermined operability irrespective of a posture of swing of the joint.

Since large swing of an joint arranged in one location can be made in multiple degrees of freedom, a tip end position is not moved due to curvature of a tip end of an instrument as in the related art (forward movements are not made following swing), it is possible to adequately observe a large range, in which a forceps can be moved, within a range of a constant (narrow) range of visual field, in which observation is made by means of an endoscope, thus enabling a treatment in wide movements of a forceps.

Also, for the joints, there is no change in wire path length even when a forceps swings. Further, freedom at the tip end side is led to freedom on the hand side to achieve transmission of drive forces through the wires. Since no change in path length is caused in spite of swing in freedom on the hand side, no external forces due to swing are applied to those wires, which extend to the tip end side, and other freedom (for example, opening and closing of the blades) is not interfered with.

While the embodiment has been described, the invention is not limited thereto and it is apparent to those skilled in the art that the invention is susceptible to various changes and modifications within the sprit of the invention and the scope of the appended claims.

1. A surgical instrument comprising a tip end joint part having an openable and closeable gripper, an operating part including a hand grip to be grasped by a palm and a plurality of operating dials, and an arm part that accommodates a wire for cooperation of actions of the operating part and the tip end joint part, and
wherein a first operating dial is arranged above the hand grip and on an upper inclined surface of the operating part and second and third operating dials are arranged above the hand grip and on a front surface of the operating part, and
wherein the first operating dial is operated by a thumb and the second operating dial is operated by a forefinger whereby the tip end joint part is operated vertically and operated laterally to perform a swinging action, and
the third operating dial is operated by a forefinger whereby the tip end gripper is operated to open and close.

2. A surgical instrument according to claim 1, wherein the first operating dial is operated by a thumb to move forward and rearward along the arm part whereby the tip end joint part is moved vertically,
the second operating dial is operated by a forefinger to move laterally whereby the tip end joint part is moved laterally, and
the third operating dial is operated by a forefinger to move the tip end gripper laterally whereby the tip end joint part is operated to open and close.

3. A surgical instrument according to claim 1, wherein a pair of rolling contact means for rolling contact are provided on the tip end joint part, a pulley is provided on one of the pair of rolling contact means, and the gripper is mounted to the pulley,
wherein the operating part comprises a projection (or a pulley) provided on the first operating dial and two rotating shafts that rotate in cooperation with rotations of the second and third operating dials, and
wherein a wire is stretched between the other of the pair of rolling contact means and the projection (or a pulley), and
a wire is stretched between the pulley of the one of the pair of rolling contact means and the two rotating shafts.

4. A surgical instrument according to claim 2, wherein a pair of rolling contact means for rolling contact are provided on the tip end joint part, a pulley is provided on one of the pair of rolling contact means, and the gripper is mounted to the pulley,
wherein the operating part comprises a projection (or a pulley) provided on the first operating dial and two rotating shafts that rotate in cooperation with rotations of the second and third operating dials, and
wherein a wire is stretched between the other of the pair of rolling contact means and the projection (or a pulley), and
a wire is stretched between the one of the pair of rolling contact means and the two rotating shafts.

5. A surgical instrument according to claim 1, further comprising a holder mounted above the hand grip and on a right or a left side of an upper portion of the operating part to bring the surgical instrument into contact with a back of a hand and to have a palm stabilized between the holder and the operating part.

6. A surgical instrument according to claim 2, further comprising a holder mounted above the hand grip and on a right or a left side of an upper portion of the operating part to bring the surgical instrument into contact with a back of a hand and to have a palm stabilized between the holder and the operating part.

7. A surgical instrument according to claim 3, wherein the second and third operating dials slide between a position, in which they are pushed against an elastic bias in a direction along a central axis of the operating part, and a position, in which they are elastically biased and not pushed, and
torque of the second and third operating dials is not transmitted to the two rotating shafts in the position, in which the dials are not pushed.

8. A surgical instrument comprising a tip end joint part having an openable and closeable gripper, an operating part including a hand grip to be grasped by a palm and a plurality of operating dials, and an arm part that accommodates a wire for cooperation of actions of the operating part and the tip end joint part, and
wherein the operating part is shaped to be substantially elliptical in cross section, first and second operating dials are arranged on an inclined surface formed on an upper portion of the hand grip on this side of the operating part, and a third operating dial is arranged on the upper portion of the hand grip on an opposite side to this side, and
wherein the first and second operating dials are operated by a thumb to move the tip end joint part vertically and laterally to have the tip end joint part swing, and
the third operating dial is operated by a forefinger whereby the tip end gripper is operated to open and close.

9. A surgical instrument according to claim 8, wherein the first operating dial is arranged centrally lateral on the inclined surface on this side, and
the second operating dial is arranged on a left or a right end of the inclined surface on this side,
wherein the first operating dial is operated by a thumb to move forward and rearward along the arm part whereby the tip end joint part is moved vertically, and the second operating dial is operated by a thumb to move lateral whereby the tip end joint part is moved laterally, and
wherein the third operating dial is operated by a forefinger to move laterally whereby the tip end joint part is operated to open and close.

10. A surgical instrument according to claim 8, wherein the first operating dial is arranged centrally lateral on the inclined surface on this side, and
the second operating dial is arranged on a left or a right end of the inclined surface on this side,
wherein the first operating dial is operated by a thumb to move forward and rearward along the arm part whereby the tip end joint part is moved vertically, and the second operating dial is operated by a thumb to move laterally whereby the tip end joint part is moved laterally, and
wherein the third operating dial is operated by a forefinger to move forward and rearward along the arm part whereby the tip end joint part is operated to open and close.

11. A surgical instrument according to claim 8, wherein a projection is provided on a lower portion of the third operating dial to define a hand grip position by a palm, a middle finger, a ring finger, and a fifth finger.

12. A surgical instrument according to claim 9, wherein a projection is provided on a lower portion of the third operating dial to define a hand grip position by a palm, a middle finger, a ring finger, and a fifth finger.

13. A surgical instrument according to claim 10, wherein a projection is provided on a lower portion of the third operating dial to define a hand grip position by a palm, a middle finger, a ring finger, and a fifth finger.

14. A surgical instrument according to claim 8, wherein a pair of rolling contact means for rolling contact are provided on the tip end joint part, a pulley is provided on one of the pair of rolling contact means, and the gripper is mounted to the pulley,
wherein the operating part comprises a projection provided on the first operating dial and two rotating shafts that rotate in cooperation with rotations of the second and third operating dials, and
wherein a wire is stretched between the other of the pair of rolling contact means and the projection, and
a wire is stretched between the pulley and the two rotating shafts.

15. A surgical instrument according to claim 8, wherein the second and third operating dials slide between a position, in which they are pushed against an elastic bias in a direction long a central axis of the operating part, and a position, in which they are elastically biased and not pushed, and
torque of the second and third operating dials is not transmitted to the two rotating shafts in the position, in which the dials are not pushed.
